(19) United States
(12) Patent Application Publication
(10) Pub. No.: US 2018/0216183 A1
(43) Pub. Date: Aug. 2, 2018

(54) HIGH CONCENTRATION REAGENTS FOR SAMPLE PREPARATION IN SMALL WELL FORMAT

(71) Applicant: Counsyl, Inc., South San Francisco, CA (US)

(72) Inventors: Henry H. Lai, San Francisco, CA (US); Clement S. Chu, San Francisco, CA (US)

(73) Assignee: Counsyl, Inc., South San Francisco, CA (US)

(21) Appl. No.: 15/881,921

(22) Filed: Jan. 29, 2018

Related U.S. Application Data
(60) Provisional application No. 62/452,886, filed on Jan. 31, 2017.

(51) Int. Cl. C12Q 1/6876; C12Q 1/6869; C40B 40/06

(52) U.S. Cl. CPC C12Q 1/6876; C12Q 1/6869; C40B 40/06; C12Y 207/01078; C12Q 2527/146; C12Q 2527/125; C12Q 2527/137; C12Q 2527/143

(57) ABSTRACT

Disclosed are high concentration reagents for use in preparing DNA samples in low volume reactions. Such reagents include, for example, DNA end repair buffers for use in low volume DNA blunting and phosphorylating reactions, DNA adenylating buffers for use in a low volume DNA adenylating reaction, and DNA ligation buffers for use in low volume DNA adaptor ligation reactions with adaptors. Also disclosed are customized reagent plates and kits containing one or more of these low volume buffers for use in low volume DNA blunting, phosphorylating, adenylating, and ligation reactions. Methods of using the high concentration reagents (low volume buffers) and the customized reagent plates for preparing DNA sequencing libraries in low volume reactions are also disclosed.

### CROSS-REFERENCE TO RELATED APPLICATIONS

This application claims priority benefit to U.S. Provisional Patent Application No. 62/452,886, filed on Jan. 31, 2017, entitled “HIGH CONCENTRATION REAGENTS FOR SAMPLE PREPARATION IN SMALL WELL FORMAT,” the content of which is hereby incorporated by reference in its entirety for all purposes.

### TECHNICAL FIELD

The present disclosure provides, inter alia, high concentration reagents for use in preparing DNA samples in low volume reactions, customized reagent plates and kits containing one or more of these low volume buffers, and methods of using the high concentration reagents (low volume buffers) and the customized reagent plates for preparing DNA sequencing libraries in low volume reactions.

### BACKGROUND

DNA library preparation is a process of adding unique sequences onto the ends of DNA with the ultimate goal of yielding DNA that is competent for sequencing. While there are many variations of library preparation methods that are available in the field, the fundamentals have not changed.

For example, in general, the first step in library preparation involves repairing DNA fragment overhangs caused by fragmentation using T4 DNA polymerase. T4 DNA polymerase is an enzyme that has a 3′-5′ exonuclease motif that allows it to chew back 3′ overhangs. Additionally, 5′ overhangs are filled in the presence of free deoxynucleotides. The final results are fragmented DNA with blunted ends. Next, the 5′ ends of the blunted DNA fragments are phosphorylated with T4 polynucleotide kinase (T4 PNK). T4 PNK catalyzes the transfer and exchange of Pi from ATP to the 5′ hydroxyl terminal of the polynucleotide. A typical end repair and phosphorylation step is illustrated in FIG. 1.

The second step of library preparation is called A-tailing. A single adenine is added to the 3′ ends of the blunted fragments by any polymerases that lack a 3′ to 5′ exonuclease capabilities. A typical A-tailing step is illustrated in FIG. 2.

The last step of library preparation is ligation. Adapters containing unique sequences of interest are ligated. T4 Ligase drives the ligation reaction in the presence of ATP. T4 ligase is an enzyme that facilitates the joining of DNA strands together by catalyzing the formation of a phosphodiester bond using ATP as a cofactor. A typical ligation step is illustrated in FIG. 3.

In early revisions of the library preparation process, a SPRIwork DNA cleanup is performed between each step to remove salts, enzymes, and other contaminants. However, every instance of a SPRIwork cleanup can cause a loss of 10-15% of the DNA.

Current technologies of library preparation have adopted a “one pot” system where no cleanup steps are required until after the ligation step. Typical “one pot” systems are illustrated in FIG. 4A and FIG. 4B. These types of “one pot” systems typically have high reaction volumes and require a thermocycler, which makes it difficult to automate the process in a 384 well system. For instance, 384 well PCR plates typically have a maximum volume capacity of 35 uL and non-PCR 384 well plates typically can accommodate up to 120 uL.

The present invention is directed to overcoming these and other deficiencies in the art.

### SUMMARY OF THE INVENTION

Disclosed are high concentration reagents for use in preparing DNA samples in low volume reactions. Such reagents include, for example, DNA end repair buffers for use in low volume DNA blunting and phosphorylating reactions, DNA adenylating buffers for use in a low volume DNA adenylating reaction, and DNA ligation buffers for use in low volume DNA adaptor ligation reactions with adaptors. Also disclosed are customized reagent plates and kits containing one or more of these low volume buffers for use in low volume DNA blunting, phosphorylating, adenylating, and ligation reactions. Methods of using the high concentration reagents (low volume buffers) and the customized reagent plates for preparing DNA sequencing libraries in low volume reactions are also disclosed.

One advantage of the various aspects of the present disclosure is to provide DNA library preparation reagents and processes that do not require the use of a thermocycler, and that require reaction volumes that are low enough to be performed in an automated fashion, including, without limitation, in a 384 well plate, therefore making it easy for high throughput automation. A schematic of one embodiment of the DNA library preparation process provided by the present disclosure is illustrated in FIG. 5, which shows the following sequential steps: (i) an End Repair step (e.g., DNA blunting and phosphorylating reactions); (ii) a Cleanup step; (iii) an A-Tail step (e.g., adenylating reaction); (iv) a Ligation step (e.g., ligation reaction); and (v) a Cleanup step.

In one aspect, the present disclosure provides a DNA end repair buffer for use in low volume DNA blunting and phosphorylating reactions. The DNA end repair buffer comprises a high concentration DNA end repair buffer mixture. In some embodiments, the high concentration DNA end repair buffer mixture comprises: (i) deoxynucleoside triphosphates at a concentration ranging from 1 mM to 2.5 mM; (ii) Tris-HCl at a concentration ranging from 150 mM to 450 mM at a pH of 7.5 to 8.0; (iii) NaCl at a concentration ranging from 60 mM to 300 mM; (iv) MgCl2 at a concentration ranging from 6 mM to 60 mM; (v) DTT at a concentration ranging from 6 mM to 30 mM; and (vi) ATP at a concentration ranging from 6 mM to 15 mM. The high concentration DNA end repair buffer mixture, when provided at a volume ranging from 2.5 μL to 5 μL, is suitable for performing low volume blunting and phosphorylating reactions of sample DNA fragments with a mixture of end repair enzymes in a single container, where the sample of DNA fragments is provided at a volume ranging from 10 μL to 20 μL. The mixture of end repair enzymes comprises a DNA blunting enzyme at a concentration ranging from 0.2 U/μL to 1.0 U/μL and a DNA phosphorylating enzyme at a concentration ranging from 2.0 U/μL to 5.0 U/μL, said mixture of end repair enzymes being provided at a volume ranging from 2.5 μL to 5 μL.

In another aspect, the present disclosure provides a kit for use in low volume DNA blunting and phosphorylating reactions. The kit comprises the DNA end repair buffer of the present disclosure and a mixture of end repair enzymes comprising a DNA blunting enzyme at a concentration ranging from 0.2 U/μL to 1 U/μL and a DNA phosphorylating enzyme at a concentration ranging from 2 U/μL to 5 U/μL.

In another aspect, the present disclosure provides a DNA adenylating buffer for use in a low volume DNA adenylating reaction. The DNA adenylating buffer comprises a high concentration DNA adenylating buffer mixture. In one embodiment, the high concentration DNA adenylating buffer mixture comprises: (i) Tris-HCl at a concentration ranging from 10 mM to 100 mM at a pH of 7.5 to 8.5; (ii) NaCl at a concentration ranging from 10 mM to 50 mM; (iii) MgCl2 at a concentration ranging from 1 mM to 10 mM; (iv) DTT at a concentration ranging from 1 mM to 5 mM; (v) dATP at a concentration ranging from 0.1 mM to 0.5 mM; and (vi) Klenow fragment at a concentration ranging from 1 U/μL to 10 U/μL. The high concentration DNA adenylating buffer mixture, when provided at a volume ranging from 5 μL to 20 μL, is suitable for performing low volume adenylating reactions of sample DNA fragments in a single container.

In another aspect, the present disclosure provides a kit for use in low volume DNA blunting, phosphorylating, and adenylating reactions. The kit comprises the DNA end repair buffer of the present disclosure; a mixture of end repair enzymes comprising a DNA blunting enzyme at a concentration ranging from 0.2 U/μL to 1 U/μL and a DNA phosphorylating enzyme at a concentration ranging from 2 U/μL to 5 U/μL; and the DNA adenylating buffer of the present disclosure.

In another aspect, the present disclosure provides a DNA ligation buffer for use in low volume DNA adaptor ligation reactions with adaptors. The DNA ligation buffer comprises a high concentration DNA ligation buffer mixture. In one embodiment, the high concentration DNA ligation buffer mixture comprises: (i) Tris-HCl at a concentration ranging from 25 mM to 250 mM at a pH of 7.5 to 8.0; (ii) MgCl2 at a concentration ranging from 2.5 mM to 25 mM; (iii) DTT at a concentration ranging from 2.5 mM to 12.5 mM; (iv) ATP at a concentration ranging from 1.25 mM to 6.25 mM; and (v) PEG 6000 at a concentration ranging from 10 percent to 25 percent. The high concentration DNA ligation buffer mixture, when provided at a volume ranging from 10 μL to 20 μL, is suitable for performing low volume adaptor ligation reactions of sample DNA fragments with a mixture of ligation enzymes at a concentration ranging from 80 c. U/μL to 200 c. U/μL, said mixture of ligation enzymes being provided at a volume ranging from 2.5 μL to 5 μL, and said adapters being provided at a volume ranging from 2.5 μL to 5 μL.

In another aspect, the present disclosure provides a kit for use in low volume DNA ligation reactions. The kit comprises the DNA ligation buffer of the present disclosure and a mixture of ligation enzymes at a concentration ranging from 80 c. U/μL to 200 c. U/μL.

In another aspect, the present disclosure provides a kit for use in low volume DNA blunting, phosphorylating, adenylating, and ligation reactions. The kit comprises: the DNA end repair buffer of the present disclosure; a mixture of end repair enzymes comprising a DNA blunting enzyme at a concentration ranging from 0.2 U/μL to 1 U/μL and a DNA phosphorylating enzyme at a concentration ranging from 2 U/μL to 10 U/μL; a DNA adenylating buffer of the present disclosure; a DNA ligation buffer of the present disclosure; and a mixture of ligation enzymes.

In another aspect, the present disclosure provides a method for preparing a DNA sequencing library. The method comprises performing the following reactions: an end repair reaction; an adenylating reaction; and a ligation reaction, thereby yielding a DNA sequencing library comprising DNA fragments each having a 3′-end and a 5′-end, the DNA fragments having synthetic DNA adapters joined to each of the 3′-ends and 5′-ends of the DNA fragments, and wherein said method does not require a thermocycler. The end repair reaction comprises mixing a sample of DNA fragments with a high concentration DNA end repair buffer and a mixture of end repair enzymes in a single container at a total volume ranging from 15 μL to 30 μL thereby performing low volume blunting and phosphorylating reactions of the DNA fragments to yield end-repaired DNA fragments. The adenylating reaction comprises performing dA-tailing of the end-repaired DNA fragments in the single container by subjecting the contents of the single container to a high concentration DNA adenylating buffer, said high concentration DNA adenylating buffer being provided at a volume ranging from 5 μL to 20 μL thereby yielding end-repaired and dA-tailed DNA fragments in the single container. The ligation reaction comprises ligating the end-repaired and dA-tailed DNA fragments to DNA adapters by introducing into the single container a high concentration DNA ligation buffer at a volume ranging from 10 μL to 20 μL, a mixture of ligation enzymes at a volume ranging from 2.5 μL to 5 μL, and a mixture of DNA adapters at a volume ranging from 2.5 μL to 5 μL. In one embodiment, the method of the present disclosure further comprises at least one cleaning step conducted in the single container, said cleaning step selected from: (i) a first cleaning step performed between the end repair reaction step and the adenylating reaction step; and/or (ii) a second cleaning step performed after the ligation reaction step.

In another aspect, the present disclosure provides a reagent plate for use in low volume DNA blunting and phosphorylating reactions. As used herein, this reagent plate is referred to as the “first reagent plate.” The reagent plate has at least one well containing a DNA end repair buffer for use in said low volume DNA blunting and phosphorylating reactions. In one embodiment, the DNA end repair buffer comprises a high concentration DNA end repair buffer mixture, where the high concentration DNA end repair buffer mixture comprises: (i) deoxynucleoside triphosphates at a concentration ranging from 1 mM to 2.5 mM; (ii) Tris-HCl at a concentration ranging from 150 mM to 450 mM at a pH of 7.5 to 8.0; (iii) NaCl at a concentration ranging from 60 mM to 300 mM; (iv) MgCl2 at a concentration ranging from 6 mM to 60 mM; (v) DTT at a concentration ranging from 6 mM to 30 mM; and (vi) ATP at a concentration ranging from 6 mM to 15 mM, where said high concentration DNA end repair buffer mixture, when provided at a volume ranging from 2.5 μL to 5 μL, is suitable for performing low volume blunting and phosphorylating reactions of sample DNA fragments with a mixture of end repair enzymes in a single container, wherein said sample of DNA fragments is provided at a volume ranging from 10 μL to 20 μL. The mixture of end repair enzymes comprises a DNA blunting enzyme at a concentration ranging from 0.2 U/μL, to 1.0 U/μL, and a DNA phosphorylating enzyme at a concentration ranging from 2.0 U/μL to 5.0 U/μL, said mixture of end repair enzymes being provided at a volume ranging from 2.5 μL to 5 μL.

In another aspect, the present disclosure provides a kit for use in low volume DNA blunting and phosphorylating reactions. The kit comprises a first reagent plate of the present disclosure and a mixture of end repair enzymes comprising a DNA blunting enzyme at a concentration ranging from 0.2 U/μL, to 1 U/μL and a DNA phosphorylating enzyme at a concentration ranging from 2 U/μL to 5 U/μL.

In another aspect, the present disclosure provides a reagent plate for use in low volume DNA adenylating reactions. As used herein, this reagent plate is referred to as the “second reagent plate.” The reagent plate has at least one well containing a DNA adenylating buffer for use in said low volume DNA adenylating reactions. In one embodiment, the DNA adenylating buffer comprises a high concentration DNA adenylating buffer mixture comprising: (i) Tris-HCl at a concentration ranging from 10 mM to 100 mM at a pH of 7.5 to 8.5; (ii) NaCl at a concentration ranging from 10 mM to 50 mM; (iii) MgCl2 at a concentration ranging from 1 mM to 10 mM; (iv) DTT at a concentration ranging from 1 mM to 5 mM; (v) dATP at a concentration ranging from 0.1 mM to 0.5 mM; and (vi) Klenow fragment at a concentration ranging from 1 U/μL to 10 U/μL. The high concentration DNA adenylating buffer mixture, when provided at a volume ranging from 5 μL to 20 μL, is suitable for performing low volume adenylating reactions of sample DNA fragments in a single container.

In another aspect, the present disclosure provides a kit for use in low volume DNA blunting, phosphorylating, and adenylating reactions. The kit comprises: a first reagent plate of the present disclosure; a mixture of end repair enzymes comprising a DNA blunting enzyme at a concentration ranging from 0.2 U/μL to 1 U/μL and a DNA phosphorylating enzyme at a concentration ranging from 2 U/μL to 5 U/μL; and a second reagent plant of the present disclosure.

In another aspect, the present disclosure provides a reagent plate for use in low volume DNA adaptor ligation reactions with adaptors. As used herein, this reagent plate is referred to as the “third reagent plate.” The reagent plate has at least one well containing a DNA ligation buffer for use in said low volume DNA adaptor ligation reactions with adaptors. In one embodiment, the DNA ligation buffer comprises a high concentration DNA ligation buffer mixture comprising: (i) Tris-HCl at a concentration ranging from 25 mM to 250 mM at a pH of 7.5 to 8.0; (ii) MgCl2 at a concentration ranging from 2.5 mM to 25 mM; (iii) DTT at a concentration ranging from 2.5 mM to 12.5 mM; (iv) ATP at a concentration ranging from 1.25 mM to 6.25 mM; and (v) PEG 6000 at a concentration ranging from 10 percent to 25 percent. The high concentration DNA ligation buffer mixture, when provided at a volume ranging from 10 μL to 20 μL, is suitable for performing low volume adaptor ligation reactions of sample DNA fragments with a mixture of ligation enzymes at a concentration ranging from 80 c. U/μL to 200 c. U/μL, said mixture of ligation enzymes being provided at a volume ranging from 2.5 μL to 5 μL, and said adapters being provided at a volume ranging from 2.5 μL to 5 μL.

In another aspect, the present disclosure provides a kit for use in low volume DNA ligation reactions. The kit comprises a third reagent plate of the present disclosure and a mixture of ligation enzymes at a concentration ranging from 80 c. U/μL to 200 c. U/μL.

In another aspect, the present disclosure provides a kit for use in low volume DNA blunting, phosphorylating, adenylating, and ligation reactions. The kit comprises: a first reagent plate of the present disclosure; a mixture of end repair enzymes comprising a DNA blunting enzyme at a concentration ranging from 0.2 U/μL to 1 U/μL and a DNA phosphorylating enzyme at a concentration ranging from 2 U/μL to 10 U/μL; a second reagent plate of the present disclosure; a third reagent plate of the present disclosure; and a mixture of ligation enzymes.

Other objects, features and advantages of the present invention will become apparent from the following detailed description. It should be understood, however, that the detailed description and specific examples, while indicating preferred embodiments of the invention, are given by way of illustration only, since various changes and modifications within the scope and spirit of the invention will become apparent to one skilled in the art from this detailed description.

### Incorporation by Reference

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

For the purpose of illustrating aspects of the present invention, there are depicted in the drawings certain embodiments of the invention. However, the invention is not limited to the precise arrangements and instrumentalities of the embodiments depicted in the drawings. Further, if provided, like reference numerals contained in the drawings are meant to identify similar or identical elements.

The file of this patent contains at least one drawing in color. Copies of this patent or patent publication with color drawing(s) will be provided by the Office upon request and payment of the necessary fee.

FIG. 1 depicts a typical end repair and phosphorylation step used in DNA library preparation processes.

FIG. 2 depicts a typical A-tailing step used in DNA library preparation processes.

FIG. 3 depicts a typical ligation step used in DNA library preparation processes.

FIG. 4A depicts a first example of a “one pot” system used in DNA library preparation processes.

FIG. 4B depicts a second example of a “one pot” system used in DNA library preparation processes.

FIG. 5 is a schematic of one embodiment of the DNA library preparation process provided by the present disclosure.

FIG. 6 is a schematic of another embodiment of the DNA library preparation process provided by the present disclosure.

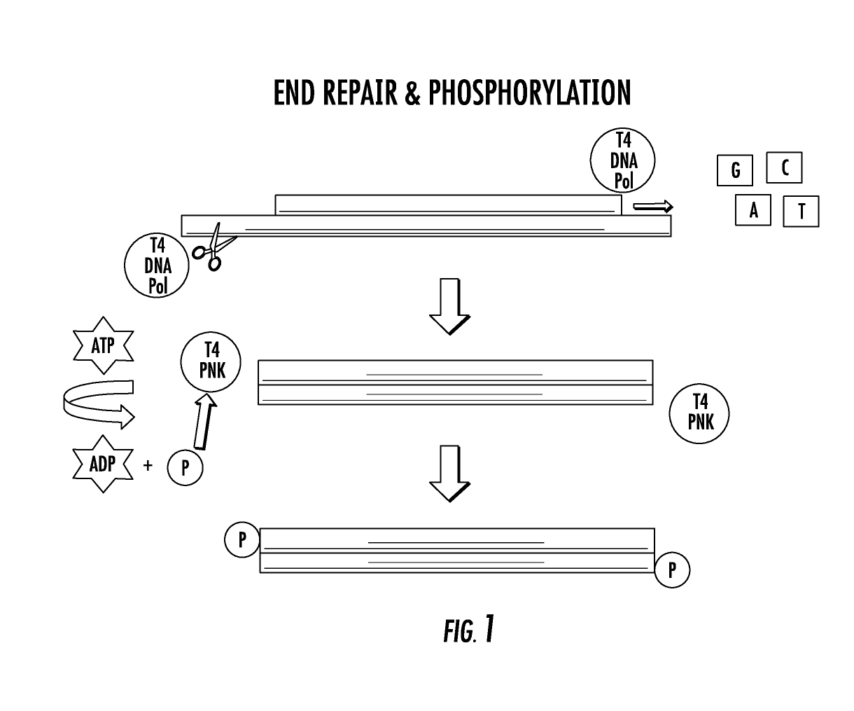

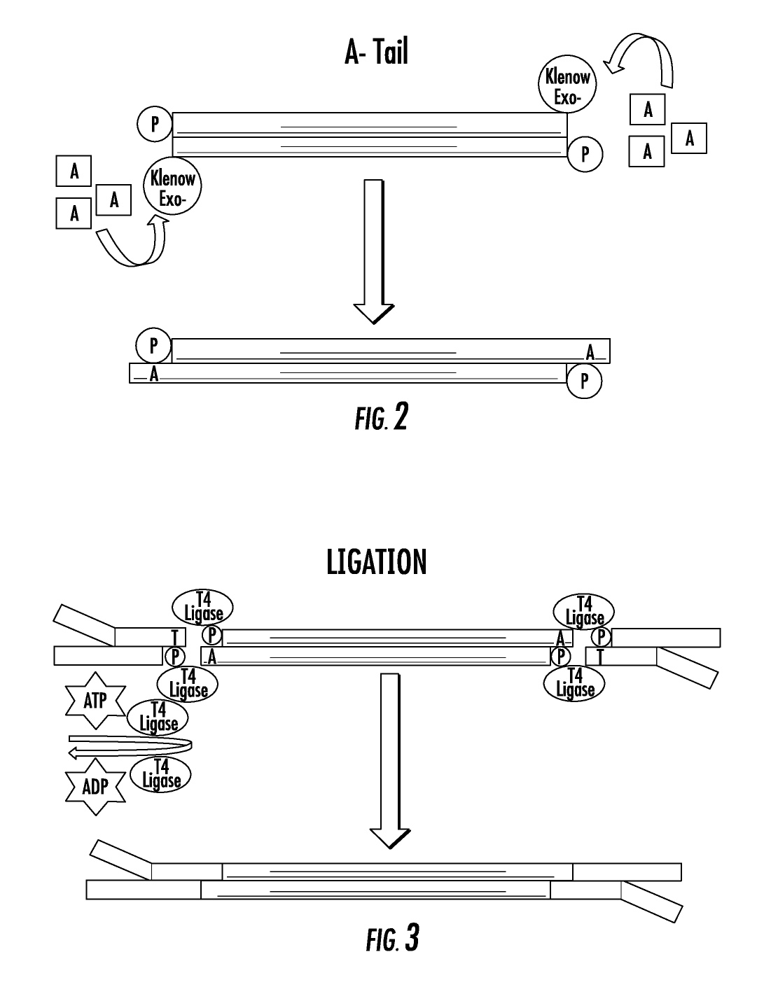

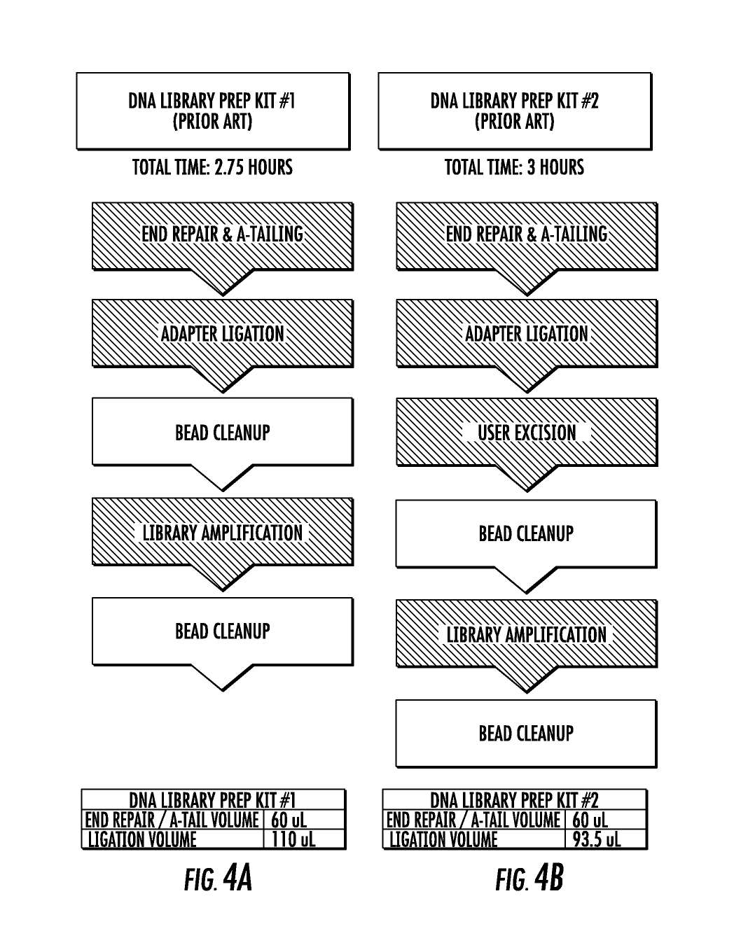

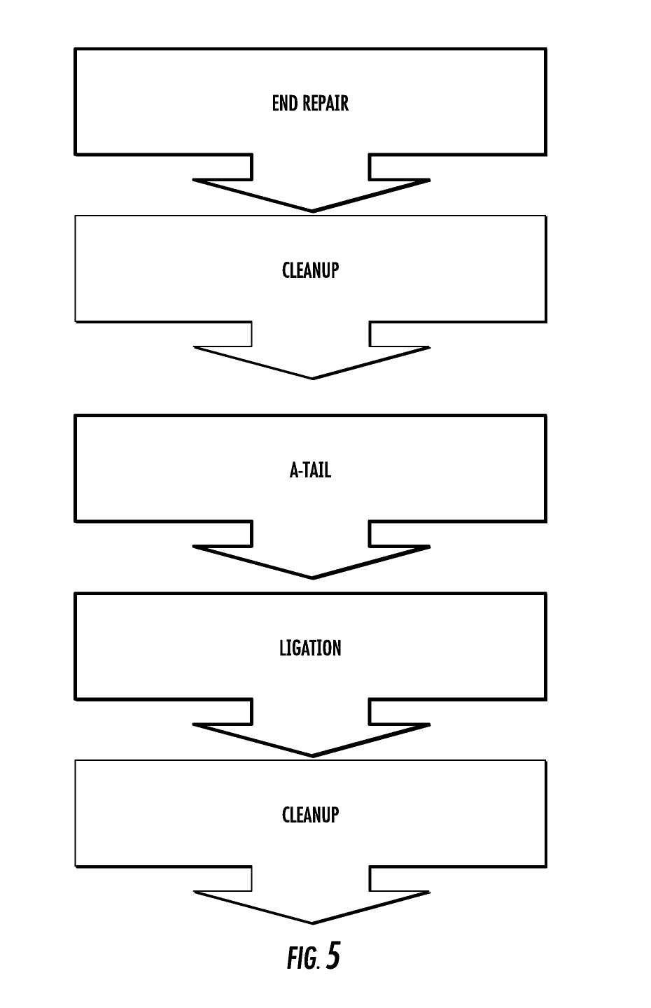

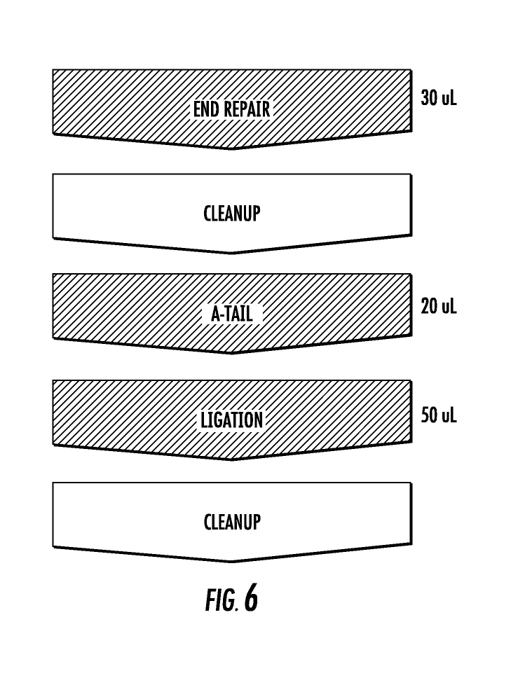

### DETAILED DESCRIPTION

Provided herein are, inter alia, high concentration reagents (also referred to herein as low volume buffers) for use in preparing DNA samples in low volume reactions, customized reagent plates and kits containing one or more of these low volume buffers, and methods of using the high concentration reagents and the customized reagent plates for preparing DNA sequencing libraries in low volume reactions.

As used herein, the term “low volume” refers to the volume of liquid needed to perform the various low volume reactions described herein to prepare DNA libraries. The term “low volume” is meant to show the unique advantages of the high concentration reagents (also referred to herein as “low volume buffers”) of the present disclosure over prior art reagents used for DNA library preparation. Specifically, prior art reagents require larger reaction volumes than the low volume buffers of the present disclosure. Because the high concentration reagents of the present disclosure require relatively lower volume DNA library preparation reactions than reagents in the prior art, the reagents of the present disclosure enable the use of small reaction wells than the prior art. For example, in certain embodiments, the high concentration reagents of the present disclosure enable the use of a microwell of a standard 384 well microplate to perform the DNA library preparation reactions as described herein. As set forth herein, such low volume reactions enable the preparation of DNA libraries without the need for using a thermocycler, and further make it practical for high throughput automation. The term “low volume” can include, without limitation, volumes of less than about 90 μL, 80 μL, 70 μL, 60 μL, 50 μL, 40 μL, 30 μL, 20 μL, or less.

The high concentration reagents, customized reagent plates, kits, and methods of the present disclosure can be used to prepare DNA libraries from any source of DNA. The DNA libraries prepared in accordance with the present disclosure can further be used for other downstream assays and analytics, including, without limitation, DNA sequencing. The DNA libraries prepared in accordance with the present disclosure can also be used to provide target polynucleotides for various uses in the broad field of biotechnology.

The terms “polynucleotide,” “nucleotide,” “nucleotide sequence,” “nucleic acid,” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, intergenic DNA, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), small nucleolar RNA, ribozymes, cDNA, recombinant polynucleotides, branched polynucleotides, plasmids, vectors, isolated DNA of any sequence, isolated RNA of any sequence, nucleic acid probes, adapters, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component, tag, reactive moiety, or binding partner. Polynucleotide sequences, when provided, are listed in the 5′ to 3′ direction, unless stated otherwise.

As used herein, the term “target polynucleotide” refers to a nucleic acid molecule or polynucleotide in a population of nucleic acid molecules having a target sequence of interest. This can include, without limitation, sequences to which one or more oligonucleotides are designed to hybridize. In some embodiments, a target sequence uniquely identifies a sequence derived from a sample, such as a particular genomic, mitochondrial, bacterial, viral, or RNA (e.g. mRNA, miRNA, primary miRNA, or pre-miRNA) sequence. In some embodiments, a target sequence is a common sequence shared by multiple different target polynucleotides, such as a common adapter sequence joined to different target polynucleotides. “Target polynucleotide” may be used to refer to a double-stranded nucleic acid molecule comprising a target sequence on one or both strands, or a single-stranded nucleic acid molecule comprising a target sequence, and may be derived from any source of or process for isolating or generating nucleic acid molecules. A target polynucleotide may comprise one or more (e.g. 1, 2, 3, 4, 5, 6, 7, 8, 9, 10, or more) target sequences, which may be the same or different. In general, different target polynucleotides comprise different sequences, such as one or more different nucleotides or one or more different target sequences.

“Hybridization” and “annealing” refer to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of a PCR, or the enzymatic cleavage of a polynucleotide by a ribozyme. A first sequence that can be stabilized via hydrogen bonding with the bases of the nucleotide residues of a second sequence is said to be “hybridizable” to the second sequence. In such a case, the second sequence can also be said to be hybridizable to the first sequence.

In general, a “complement” of a given sequence is a sequence that is fully complementary to and hybridizable to the given sequence. In general, a first sequence that is hybridizable to a second sequence or set of second sequences is specifically or selectively hybridizable to the second sequence or set of second sequences, such that hybridization to the second sequence or set of second sequences is preferred (e.g. thermodynamically more stable under a given set of conditions, such as stringent conditions commonly used in the art) to hybridization with non-target sequences during a hybridization reaction. Typically, hybridizable sequences share a degree of sequence complementarity over all or a portion of their respective lengths, such as between 25%-100% complementarity, including at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, and 100% sequence complementarity.

The term “hybridized” as applied to a polynucleotide refers to a polynucleotide in a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi-stranded complex, a single self-hybridizing strand, or any combination of these. The hybridization reaction may constitute a step in a more extensive process, such as the initiation of a PCR reaction, ligation reaction, sequencing reaction, or cleavage reaction.

The practice of the present invention employs, unless otherwise indicated, conventional techniques of immunology, biochemistry, chemistry, molecular biology, microbiology, cell biology, genomics and recombinant DNA, which are within the skill of the art. See e.g. Sambrook, Fritsch and Maniatis, MOLECULAR CLONING: A LABORATORY MANUAL, 2nd edition (1989); CURRENT PROTOCOLS 1N MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

### I. High Concentration Reagents for Sample Preparation in Small Well Format

The present disclosure provides high concentration reagents for use in preparing DNA samples in low volume reactions. As discussed in more detail herein, such reagents include, for example, DNA end repair buffers for use in low volume DNA blunting and phosphorylating reactions, DNA adenylating buffers for use in a low volume DNA adenylating reaction, and DNA ligation buffers for use in low volume DNA adaptor ligation reactions with adaptors. The present disclosure also provides kits containing one or more of these low volume buffers for use in low volume DNA blunting, phosphorylating, adenylating, and ligation reactions.

### A. DNA End Repair Buffer

In one aspect, the present disclosure provides a DNA end repair buffer for use in low volume DNA blunting and phosphorylating reactions. The DNA end repair buffer comprises a high concentration DNA end repair buffer mixture. In some embodiments, the high concentration DNA end repair buffer mixture comprises: (i) deoxynucleoside triphosphates at a concentration ranging from 1 mM to 2.5 mM; (ii) Tris-HCl at a concentration ranging from 150 mM to 450 mM at a pH of 7.5 to 8.0; (iii) NaCl at a concentration ranging from 60 mM to 300 mM; (iv) MgCl2 at a concentration ranging from 6 mM to 60 mM; (v) DTT at a concentration ranging from 6 mM to 30 mM; and (vi) ATP at a concentration ranging from 6 mM to 15 mM. The high concentration DNA end repair buffer mixture, when provided at a volume ranging from 2.5 μL to 5 μL, is suitable for performing low volume blunting and phosphorylating reactions of sample DNA fragments with a mixture of end repair enzymes in a single container, where the sample of DNA fragments is provided at a volume ranging from 10 μL to 20 μL. The mixture of end repair enzymes comprises a DNA blunting enzyme at a concentration ranging from 0.2 U/μL to 1.0 U/μL, and a DNA phosphorylating enzyme at a concentration ranging from 2.0 U/μL, to 5.0 U/μL, said mixture of end repair enzymes being provided at a volume ranging from 2.5 μL to 5 μL.

In certain embodiments, the deoxynucleoside triphosphates comprise dATP, dCTP, dTTP, and dGTP, where the concentration of dATP ranges from 1 mM to 2.5 mM, the concentration of dCTP ranges from 1 mM to 2.5 mM, the concentration of dTTP ranges from 1 mM to 2.5 mM, and the concentration of dGTP ranges from 1 mM to 2.5 mM.

In certain embodiments, the concentration of dATP is about 1.5 mM, the concentration of dCTP is about 1.5 mM, the concentration of dTTP is about 1.5 mM, and the concentration of dGTP is about 1.5 mM.

In certain embodiments, the high concentration DNA end repair buffer mixture comprises: (i) deoxynucleoside triphosphates at a concentration of about 1.5 mM; (ii) Tris-HCl at a concentration of about 300 mM at a pH of about 7.6; (iii) NaCl at a concentration of about 300 mM; (iv) MgCl₂ at a concentration of about 60 mM; (v) DTT at a concentration of about 30 mM; and (vi) ATP at a concentration of about 6 mM.

In certain embodiments, the high concentration DNA end repair buffer mixture, when provided at a volume of about 5 μL, is suitable for performing low volume blunting and phosphorylating reactions containing 20 μL of the sample DNA fragments and 5 μL of the mixture of end repair enzymes, thereby resulting in a total volume of 30 μL during the performance of the low volume blunting and phosphorylating reactions in the single container.

In certain embodiments, the 5 μL mixture of end repair enzymes comprises the DNA blunting enzyme at a concentration of about 0.6 U/μL and the DNA phosphorylating enzyme at a concentration of about 2 U/μL.

In certain embodiments, the DNA blunting enzyme is selected from the group consisting of T4 DNA polymerase, T7 DNA polymerase, and DNA Polymerase I, Large (Klenow) Fragment.

In certain embodiments, the DNA phosphorylating enzyme is selected from the group consisting of T4 polynucleotidekinase, and variants thereof.

In certain embodiments, the buffer further comprises at least one component selected from the group consisting of Triton X-100, glycerol, NP 40, EDTA, Tween 20, and variants thereof.

In certain embodiments, the single container is a microwell of a microplate including any one of 96, 384, 1536, 3456 or 9600 microwells.

In certain embodiments, the DNA end repair buffer is suitable for high throughput automated low volume DNA blunting and phosphorylating reactions.

In certain embodiments, the DNA end repair buffer is suitable for low volume DNA blunting and phosphorylating reactions that do not require a thermocycler.

In another aspect, the present disclosure provides a kit for use in low volume DNA blunting and phosphorylating reactions. The kit comprises the DNA end repair buffer of the present disclosure and a mixture of end repair enzymes comprising a DNA blunting enzyme at a concentration ranging from 0.2 U/μL to 1 U/μL and a DNA phosphorylating enzyme at a concentration ranging from 2 U/μL to 5 U/μL.

### B. DNA Adenylating Buffer

In another aspect, the present disclosure provides a DNA adenylating buffer for use in a low volume DNA adenylating reaction. The DNA adenylating buffer comprises a high concentration DNA adenylating buffer mixture. In one embodiment, the high concentration DNA adenylating buffer mixture comprises: (i) Tris-HCl at a concentration ranging from 10 mM to 100 mM at a pH of 7.5 to 8.5; (ii) NaCl at a concentration ranging from 10 mM to 50 mM; (iii) MgCl2 at a concentration ranging from 1 mM to 10 mM; (iv) DTT at a concentration ranging from 1 mM to 5 mM; (v) dATP at a concentration ranging from 0.1 mM to 0.5 mM; and (vi) Klenow fragment at a concentration ranging from 1 U/μL to 10 U/μL. The high concentration DNA adenylating buffer mixture, when provided at a volume ranging from 5 μL to 20 μL, is suitable for performing low volume adenylating reactions of sample DNA fragments in a single container.

In certain embodiments, the high concentration DNA adenylating buffer mixture comprises: (i) Tris-HCl at a concentration of about 20 mM at a pH of about 8.0; (ii) NaCl at a concentration of about 50 mM; (iii) MgCl₂ at a concentration of about 10 mM; (iv) DTT at a concentration of about 1 mM; (v) dATP at a concentration of about 0.2 mM; and (vi) Klenow fragment at a concentration of about 0.375 U/μL.

In certain embodiments, the high concentration DNA adenylating buffer mixture, when provided at a volume of about 20 μL, is suitable for performing low volume adenylating reactions of sample DNA fragments in a single container.

In certain embodiments, the buffer further comprises at least one component selected from the group consisting of Triton X-100, glycerol, NP 40, EDTA, Tween 20, and variants thereof.

In certain embodiments, the single container is a microwell of a microplate including any one of 96, 384, 1536, 3456 or 9600 microwells.

In certain embodiments, the DNA adenylating buffer is suitable for high throughput automated low volume DNA adenylating reactions.

In certain embodiments, the DNA adenylating buffer is suitable for low volume DNA adenylating reactions that do not require a thermocycler.

In another aspect, the present disclosure provides a kit for use in low volume DNA blunting, phosphorylating, and adenylating reactions. The kit comprises the DNA end repair buffer of the present disclosure; a mixture of end repair enzymes comprising a DNA blunting enzyme at a concentration ranging from 0.2 U/μL to 1 U/μL and a DNA phosphorylating enzyme at a concentration ranging from 2 U/μL to 5 U/μL; and the DNA adenylating buffer of the present disclosure.

### C. DNA Ligation Buffer

In another aspect, the present disclosure provides a DNA ligation buffer for use in low volume DNA adaptor ligation reactions with adaptors. The DNA ligation buffer comprises a high concentration DNA ligation buffer mixture. In one embodiment, the high concentration DNA ligation buffer mixture comprises: (i) Tris-HCl at a concentration ranging from 25 mM to 250 mM at a pH of 7.5 to 8.0; (ii) MgCl2 at a concentration ranging from 2.5 mM to 25 mM; (iii) DTT at a concentration ranging from 2.5 mM to 12.5 mM; (iv) ATP at a concentration ranging from 1.25 mM to 6.25 mM; and (v) PEG 6000 at a concentration ranging from 10 percent to 25 percent. The high concentration DNA ligation buffer mixture, when provided at a volume ranging from 10 μL to 20 μL, is suitable for performing low volume adaptor ligation reactions of sample DNA fragments with a mixture of ligation enzymes at a concentration ranging from 80 c. U/μL to 200 c. U/μL, said mixture of ligation enzymes being provided at a volume ranging from 2.5 μL to 5 μL, and said adapters being provided at a volume ranging from 2.5 μL to 5 μL.

In certain embodiments, the high concentration DNA ligation buffer mixture comprises: (i) Tris-HCl at a concentration of about 50 mM at a pH of about 7.6; (ii) MgCl₂ at a concentration of about 25 mM; (iii) DTT at a concentration of about 2.5 mM; (iv) ATP at a concentration of about 5 mM; and (v) PEG 6000 at a concentration of about 17.5 percent.

In certain embodiments, the high concentration DNA ligation buffer mixture, when provided at a volume of about 20 μL, is suitable for performing low volume DNA adaptor ligation reactions containing 20 μL of the sample DNA fragments, 5 μL of the mixture of ligation enzymes, and 5 μL of the adaptors, thereby resulting in a total volume of 50 μL during the performance of the low volume adaptor ligation reactions in the single container.

In certain embodiments, the the 5 μL mixture of ligation enzymes is provided at a concentration of about 127 c. U/μL.

In certain embodiments, the ligation enzyme is selected from the group consisting of T4 DNA ligase, T3 DNA Ligase, and T7 DNA Ligase.

In certain embodiments, the buffer further comprises at least one component selected from the group consisting of Triton X-100, glycerol, NP 40, EDTA, Tween 20, and variants thereof.

In certain embodiments, the single container is a microwell of a microplate including any one of 96, 384, 1536, 3456 or 9600 microwells.

In certain embodiments, the DNA ligation buffer is suitable for high throughput automated low volume DNA adaptor ligation reactions.

In certain embodiments, the DNA ligation buffer is suitable for low volume DNA ligation reactions that do not require a thermocycler.

In another aspect, the present disclosure provides a kit for use in low volume DNA ligation reactions. The kit comprises the DNA ligation buffer of the present disclosure and a mixture of ligation enzymes at a concentration ranging from 80 c. U/μL to 200 c. U/μL.

In another aspect, the present disclosure provides a kit for use in low volume DNA blunting, phosphorylating, adenylating, and ligation reactions. The kit comprises: the DNA end repair buffer of the present disclosure; a mixture of end repair enzymes comprising a DNA blunting enzyme at a concentration ranging from 0.2 U/μL to 1 U/μL and a DNA phosphorylating enzyme at a concentration ranging from 2 U/μL to 10 U/μL; a DNA adenylating buffer of the present disclosure; a DNA ligation buffer of the present disclosure; and a mixture of ligation enzymes.

### II. Sequencing Library Preparation in Small Well Format

The present disclosure provides methods for using high concentration reagents in preparing DNA samples in low volume reactions for the purpose of preparing DNA sequencing libraries. As discussed in more detail herein, such methods can also involve the use customized reagent plates for preparing DNA sequencing libraries in low volume reactions.

In one aspect, the present disclosure provides a method for preparing a DNA sequencing library. The method comprises performing the following reactions: an end repair reaction; an adenylating reaction; and a ligation reaction, thereby yielding a DNA sequencing library comprising DNA fragments each having a 3′-end and a 5′-end, the DNA fragments having synthetic DNA adapters joined to each of the 3′-ends and 5′-ends of the DNA fragments, and wherein said method does not require a thermocycler. The end repair reaction comprises mixing a sample of DNA fragments with a high concentration DNA end repair buffer and a mixture of end repair enzymes in a single container at a total volume ranging from 15 μL to 30 μL, thereby performing low volume blunting and phosphorylating reactions of the DNA fragments to yield end-repaired DNA fragments. The adenylating reaction comprises performing dA-tailing of the end-repaired DNA fragments in the single container by subjecting the contents of the single container to a high concentration DNA adenylating buffer, said high concentration DNA adenylating buffer being provided at a volume ranging from 5 μL to 20 μL, thereby yielding end-repaired and dA-tailed DNA fragments in the single container. The ligation reaction comprises ligating the end-repaired and dA-tailed DNA fragments to DNA adapters by introducing into the single container a high concentration DNA ligation buffer at a volume ranging from 10 μL to 20 μL, a mixture of ligation enzymes at a volume ranging from 2.5 μL to 5 μL, and a mixture of DNA adapters at a volume ranging from 2.5 μL to 5 μL.

In certain embodiments, the end repair reaction is performed at a temperature ranging from 16° C. to 25° C. for a period ranging from 20 minutes to 40 minutes, where the adenylating reaction is performed at a temperature ranging from 20° C. to 37° C. for a period ranging from 20 minutes to 40 minutes, and where the ligation reaction is performed at a temperature ranging from 16° C. to 25° C. for a period ranging from 15 minutes to 30 minutes.

In one embodiment, the method of the present disclosure further comprises at least one cleaning step conducted in the single container, said cleaning step selected from: (i) a first cleaning step performed between the end repair reaction step and the adenylating reaction step; and/or (ii) a second cleaning step performed after the ligation reaction step.

In certain embodiments, the first cleaning step comprises incubating the end-repaired DNA fragments yielded from the end repair reaction step to a 2:1 bead mix-to-DNA sample ratio to yield a total volume ranging from 45 μL to 90 μL, and thereafter washing and drying the end-repaired DNA fragments bound to the beads.

In certain embodiments, the first cleaning step yields a total volume of about 90 μL of cleaned end-repaired DNA fragments when the volume of the end repair reaction is about 30 μL.

In certain embodiments, the second cleaning step comprises incubating the ligated DNA fragments yielded from the ligation reaction step to a 0.8:1 bead mix-to-DNA sample ratio to yield a total volume ranging from 36 μL to 90 μL, and thereafter washing and drying the ligated DNA fragments bound to the beads and then eluting the bead-bound ligated DNA fragments.

In certain embodiments, the second cleaning step yields a total volume of about 90 μL of cleaned ligated DNA fragments when the volume of the ligation reaction is about 50 μL.

In certain embodiments, the DNA end repair buffer comprises: a high concentration DNA end repair buffer mixture comprising: (i) deoxynucleoside triphosphates at a concentration ranging from 1 mM to 2.5 mM; (ii) Tris-HCl at a concentration ranging from 150 mM to 450 mM at a pH of 7.5 to 8.0; (iii) NaCl at a concentration ranging from 60 mM to 300 mM; (iv) MgCl2 at a concentration ranging from 6 mM to 60 mM; (v) DTT at a concentration ranging from 6 mM to 30 mM; and (vi) ATP at a concentration ranging from 6 mM to 15 mM, where said high concentration DNA end repair buffer mixture, when provided at a volume ranging from 2.5 μL to 5 μL, is suitable for performing low volume blunting and phosphorylating reactions of sample DNA fragments with a mixture of end repair enzymes in a single container, wherein said sample of DNA fragments is provided at a volume ranging from 10 μL to 20 μL, and wherein said mixture of end repair enzymes comprises a DNA blunting enzyme at a concentration ranging from 0.2 U/μL to 1.0 U/μL and a DNA phosphorylating enzyme at a concentration ranging from 2.0 U/μL to 5.0 U/μL, said mixture of end repair enzymes being provided at a volume ranging from 2.5 μL to 5 μL.

In certain embodiments, the deoxynucleoside triphosphates comprise dATP, dCTP, dTTP, and dGTP, where the concentration of dATP ranges from 1 mM to 2.5 mM, the concentration of dCTP ranges from 1 mM to 2.5 mM, the concentration of dTTP ranges from 1 mM to 2.5 mM, and the concentration of dGTP ranges from 1 mM to 2.5 mM.

In certain embodiments, the concentration of dATP is about 1.5 mM, the concentration of dCTP is about 1.5 mM, the concentration of dTTP is about 1.5 mM, and the concentration of dGTP is about 1.5 mM.

In certain embodiments, the high concentration DNA end repair buffer mixture comprises: (i) deoxynucleoside triphosphates at a concentration of about 1.5 mM; (ii) Tris-HCl at a concentration of about 300 mM at a pH of about 7.6; (iii) NaCl at a concentration of about 300 mM; (iv) MgCl2 at a concentration of about 60 mM; (v) DTT at a concentration of about 30 mM; and (vi) ATP at a concentration of about 6 mM.

In certain embodiments, the high concentration DNA end repair buffer mixture, when provided at a volume of about 5 μL, is suitable for performing low volume blunting and phosphorylating reactions containing 20 μL of the sample DNA fragments and 5 μL of the mixture of end repair enzymes, thereby resulting in a total volume of 30 μL during the performance of the low volume blunting and phosphorylating reactions in the single container.

In certain embodiments, the 5 μL mixture of end repair enzymes comprises the DNA blunting enzyme at a concentration of about 0.6 U/μL, and the DNA phosphorylating enzyme at a concentration of about 2 U/μL.

In certain embodiments, the DNA blunting enzyme is selected from the group consisting of T4 DNA polymerase, T7 DNA polymerase, and DNA Polymerase I, Large (Klenow) Fragment.

In certain embodiments, the DNA phosphorylating enzyme is selected from the group consisting of T4 polynucleotidekinase, and variants thereof.

In certain embodiments, the high concentration DNA end repair buffer further comprises at least one component selected from the group consisting of Triton X-100, glycerol, NP 40, EDTA, Tween 20, and variants thereof.

In certain embodiments, the DNA adenylating buffer comprises: a high concentration DNA adenylating buffer mixture comprising: (i) Tris-HCl at a concentration ranging from 10 mM to 100 mM at a pH of 7.5 to 8.5; (ii) NaCl at a concentration ranging from 10 mM to 50 mM; (iii) MgCl2 at a concentration ranging from 1 mM to 10 mM; (iv) DTT at a concentration ranging from 1 mM to 5 mM; (v) dATP at a concentration ranging from 0.1 mM to 0.5 mM; and (vi) Klenow fragment at a concentration ranging from 1 U/μL, to 10 U/μL, where said high concentration DNA adenylating buffer mixture, when provided at a volume ranging from 5 μL to 20 μL, is suitable for performing low volume adenylating reactions of sample DNA fragments in a single container.

In certain embodiments, the high concentration DNA adenylating buffer mixture comprises: (i) Tris-HCl at a concentration of about 20 mM at a pH of about 8.0; (ii) NaCl at a concentration of about 50 mM; (iii) MgCl2 at a concentration of about 10 mM; (iv) DTT at a concentration of about 1 mM; (v) dATP at a concentration of about 0.2 mM; and (vi) Klenow fragment at a concentration of about 0.375 U/μL.

In certain embodiments, the high concentration DNA adenylating buffer mixture, when provided at a volume of about 20 μL, is suitable for performing low volume adenylating reactions of sample DNA fragments in a single container.

In certain embodiments, the high concentration DNA adenylating buffer further comprises at least one component selected from the group consisting of Triton X-100, glycerol, NP 40, EDTA, Tween 20, and variants thereof.

In certain embodiments, the DNA ligation buffer comprises: a high concentration DNA ligation buffer mixture comprising: (i) Tris-HCl at a concentration ranging from 25 mM to 250 mM at a pH of 7.5 to 8.0; (ii) MgCl2 at a concentration ranging from 2.5 mM to 25 mM; (iii) DTT at a concentration ranging from 2.5 mM to 12.5 mM; (iv) ATP at a concentration ranging from 1.25 mM to 6.25 mM; and (v) PEG 6000 at a concentration ranging from 10 percent to 25 percent, where said high concentration DNA ligation buffer mixture, when provided at a volume ranging from 10 μL to 20 μL, is suitable for performing low volume adaptor ligation reactions of sample DNA fragments with a mixture of ligation enzymes at a concentration ranging from 80 c. U/μL to 200 c. U/μL, said mixture of ligation enzymes being provided at a volume ranging from 2.5 μL to 5 μL, and said adapters being provided at a volume ranging from 2.5 μL to 5 μL.

In certain embodiments, the high concentration DNA ligation buffer mixture comprises: (i) Tris-HCl at a concentration of about 50 mM at a pH of about 7.6; (ii) MgCl2 at a concentration of about 25 mM; (iii) DTT at a concentration of about 2.5 mM; (iv) ATP at a concentration of about 5 mM; and (v) PEG 6000 at a concentration of about 17.5 percent.

In certain embodiments, the high concentration DNA ligation buffer mixture, when provided at a volume of about 20 μL, is suitable for performing low volume DNA adaptor ligation reactions containing 20 μL of the sample DNA fragments, 5 μL of the mixture of ligation enzymes, and 5 μL of the adaptors, thereby resulting in a total volume of 50 μL during the performance of the low volume adaptor ligation reactions in the single container.

In certain embodiments, the 5 μL mixture of ligation enzymes is provided at a concentration of about 127 c. U/μL.

In certain embodiments, the ligation enzyme is selected from the group consisting of T4 DNA ligase, T3 DNA Ligase, and T7 DNA Ligase.

In certain embodiments, the high concentration DNA ligation buffer further comprises at least one component selected from the group consisting of Triton X-100, glycerol, NP 40, EDTA, Tween 20, and variants thereof.

In certain embodiments, the method is conducted in high throughput, automated, and low volume reactions.

In certain embodiments, the end repair reaction, the adenylating reaction, and the ligation reaction are performed in a microwell of a microplate, wherein the end repair reaction, the adenylating reaction, and the ligation reaction take place either in the same or different microwells of the same or different microplates.

In certain embodiments, the microplate has a physical size that conforms to an industry standard.

In certain embodiments, the microplate includes any one of 96, 384, 1536, 3456 or 9600 microwells.

In certain embodiments, the microwells are arranged in a rectangular array.

In certain embodiments, the microplate is a standard 384-microwell microplate.

In certain embodiments, the microplate is made of any one of polystyrene and polypropylene.

In certain embodiments, the microplate is sealed.

In certain embodiments, the microplate is sealed with a metallic foil.

In certain embodiments, the metallic foil is piercable.

In certain embodiments, the metallic foil can be removed by peeling.

In certain embodiments, the metallic foil is aluminum.

In certain embodiments, the microplate is labeled.

In certain embodiments, the one or more of said plurality of microwells contains a sufficient amount of said DNA end repair buffer, DNA adenylating buffer, and DNA ligation buffer to perform said end repair reaction, adenylating reaction, and ligation reaction, respectively.

### III. Customized Reagent Plates

The present disclosure provides customized reagent plates that comprise high concentration reagents for use in preparing DNA samples in low volume reactions for preparing DNA sequencing libraries. As discussed in more detail herein, such customized reagent plates comprise, for example, DNA end repair buffers for use in low volume DNA blunting and phosphorylating reactions, DNA adenylating buffers for use in a low volume DNA adenylating reaction, and DNA ligation buffers for use in low volume DNA adaptor ligation reactions with adaptors. Also described are kits comprising customized reagent plates containing one or more of the low volume buffers for use in low volume DNA blunting, phosphorylating, adenylating, and ligation reactions.

A. Reagent Plate for Use in Low Volume DNA Blunting and Phosphorylating Reactions

In one aspect, the present disclosure provides a reagent plate for use in low volume DNA blunting and phosphorylating reactions. As used herein, this reagent plate is referred to as the “first reagent plate.” The reagent plate has at least one well containing a DNA end repair buffer for use in said low volume DNA blunting and phosphorylating reactions. In one embodiment, the DNA end repair buffer comprises a high concentration DNA end repair buffer mixture, where the high concentration DNA end repair buffer mixture comprises: (i) deoxynucleoside triphosphates at a concentration ranging from 1 mM to 2.5 mM; (ii) Tris-HCl at a concentration ranging from 150 mM to 450 mM at a pH of 7.5 to 8.0; (iii) NaCl at a concentration ranging from 60 mM to 300 mM; (iv) MgCl2 at a concentration ranging from 6 mM to 60 mM; (v) DTT at a concentration ranging from 6 mM to 30 mM; and (vi) ATP at a concentration ranging from 6 mM to 15 mM, where said high concentration DNA end repair buffer mixture, when provided at a volume ranging from 2.5 μL to 5 μL, is suitable for performing low volume blunting and phosphorylating reactions of sample DNA fragments with a mixture of end repair enzymes in a single container, wherein said sample of DNA fragments is provided at a volume ranging from 10 μL to 20 μL. The mixture of end repair enzymes comprises a DNA blunting enzyme at a concentration ranging from 0.2 U/μL to 1.0 U/μL and a DNA phosphorylating enzyme at a concentration ranging from 2.0 U/μL to 5.0 U/μL said mixture of end repair enzymes being provided at a volume ranging from 2.5 μL to 5 μL.

In certain embodiments, the deoxynucleoside triphosphates comprise dATP, dCTP, dTTP, and dGTP, where the concentration of dATP ranges from 1 mM to 2.5 mM, the concentration of dCTP ranges from 1 mM to 2.5 mM, the concentration of dTTP ranges from 1 mM to 2.5 mM, and the concentration of dGTP ranges from 1 mM to 2.5 mM.

In certain embodiments, the concentration of dATP is about 1.5 mM, the concentration of dCTP is about 1.5 mM, the concentration of dTTP is about 1.5 mM, and the concentration of dGTP is about 1.5 mM.

In certain embodiments, the high concentration DNA end repair buffer mixture comprises: (i) deoxynucleoside triphosphates at a concentration of about 1.5 mM; (ii) Tris-HCl at a concentration of about 300 mM at a pH of about 7.6; (iii) NaCl at a concentration of about 300 mM; (iv) MgCl2 at a concentration of about 60 mM; (v) DTT at a concentration of about 30 mM; and (vi) ATP at a concentration of about 6 mM.

In certain embodiments, the high concentration DNA end repair buffer mixture, when provided at a volume of about 5 μL, is suitable for performing low volume blunting and phosphorylating reactions containing 20 μL of the sample DNA fragments and 5 μL of the mixture of end repair enzymes, thereby resulting in a total volume of 30 μL during the performance of the low volume blunting and phosphorylating reactions in the single container.

In certain embodiments, the 5 μL mixture of end repair enzymes comprises the DNA blunting enzyme at a concentration of about 0.6 U/μL and the DNA phosphorylating enzyme at a concentration of about 2 U/μL.

In certain embodiments, the DNA blunting enzyme is selected from the group consisting of T4 DNA polymerase, T7 DNA polymerase, and DNA Polymerase I, Large (Klenow) Fragment.

In certain embodiments, the DNA phosphorylating enzyme is selected from the group consisting of T4 polynucleotidekinase, and variants thereof.

In certain embodiments, the reagent plate further comprises at least one component selected from the group consisting of Triton X-100, glycerol, NP 40, EDTA, Tween 20, and variants thereof.

In certain embodiments, the DNA end repair buffer is suitable for high throughput automated low volume DNA blunting and phosphorylating reactions.

In certain embodiments, the DNA end repair buffer is suitable for low volume DNA blunting and phosphorylating reactions that do not require a thermocycler.

In certain embodiments, the reagent plate is a microplate comprising a plurality of microwells, wherein each microwell corresponds to a separate, single container.

In certain embodiments, the microplate has a physical size that conforms to an industry standard.

In certain embodiments, the microplate includes any one of 96, 384, 1536, 3456 or 9600 microwells.

In certain embodiments, the microwells are arranged in a rectangular array.

In certain embodiments, the microplate is a standard 384-microwell microplate.

In certain embodiments, the microplate is made of any one of polystyrene and polypropylene.

In certain embodiments, the microplate is sealed.

In certain embodiments, the microplate is sealed with a metallic foil.

In certain embodiments, the metallic foil is piercable.

In certain embodiments, the metallic foil can be removed by peeling.

In certain embodiments, the metallic foil is aluminum.

In certain embodiments, the microplate is labeled.

In certain embodiments, the one or more of said plurality of microwells contains a sufficient amount of said DNA end repair buffer to for perform said low volume DNA blunting and phosphorylation reactions.

In another aspect, the present disclosure provides a kit for use in low volume DNA blunting and phosphorylating reactions. The kit comprises a first reagent plate of the present disclosure and a mixture of end repair enzymes comprising a DNA blunting enzyme at a concentration ranging from 0.2 U/μL to 1 U/μL and a DNA phosphorylating enzyme at a concentration ranging from 2 U/μL to 5 U/μL.

B. Reagent Plate for Use in Low Volume DNA Adenylating Reactions

In another aspect, the present disclosure provides a reagent plate for use in low volume DNA adenylating reactions. As used herein, this reagent plate is referred to as the “second reagent plate.” The reagent plate has at least one well containing a DNA adenylating buffer for use in said low volume DNA adenylating reactions. In one embodiment, the DNA adenylating buffer comprises a high concentration DNA adenylating buffer mixture comprising: (i) Tris-HCl at a concentration ranging from 10 mM to 100 mM at a pH of 7.5 to 8.5; (ii) NaCl at a concentration ranging from 10 mM to 50 mM; (iii) MgCl2 at a concentration ranging from 1 mM to 10 mM; (iv) DTT at a concentration ranging from 1 mM to 5 mM; (v) dATP at a concentration ranging from 0.1 mM to 0.5 mM; and (vi) Klenow fragment at a concentration ranging from 1 U/μL to 10 U/μL. The high concentration DNA adenylating buffer mixture, when provided at a volume ranging from 5 μL to 20 μL, is suitable for performing low volume adenylating reactions of sample DNA fragments in a single container.

In certain embodiments, the high concentration DNA adenylating buffer mixture comprises: (i) Tris-HCl at a concentration of about 20 mM at a pH of about 8.0; (ii) NaCl at a concentration of about 50 mM; (iii) MgCl2 at a concentration of about 10 mM; (iv) DTT at a concentration of about 1 mM; (v) dATP at a concentration of about 0.2 mM; and (vi) Klenow fragment at a concentration of about 0.375 U/μL.

In certain embodiments, the high concentration DNA adenylating buffer mixture, when provided at a volume of about 20 μL is suitable for performing low volume adenylating reactions of sample DNA fragments in a single container.

In certain embodiments, the reagent plate further comprises at least one component selected from the group consisting of Triton X-100, glycerol, NP 40, EDTA, Tween 20, and variants thereof.

In certain embodiments, the DNA adenylating buffer is suitable for high throughput automated low volume DNA adenylating reactions.

In certain embodiments, the DNA adenylating buffer is suitable for low volume DNA adenylating reactions that do not require a thermocycler.

In certain embodiments, the reagent plate is a microplate comprising a plurality of microwells, wherein each microwell corresponds to a separate, single container.

In certain embodiments, the microplate has a physical size that conforms to an industry standard.

In certain embodiments, the microplate includes any one of 96, 384, 1536, 3456 or 9600 microwells.

In certain embodiments, the microwells are arranged in a rectangular array.

In certain embodiments, the microplate is a standard 384-microwell microplate.

In certain embodiments, the microplate is made of any one of polystyrene and polypropylene.

In certain embodiments, the microplate is sealed.

In certain embodiments, the microplate is sealed with a metallic foil.

In certain embodiments, the metallic foil is piercable.

In certain embodiments, the metallic foil can be removed by peeling.

In certain embodiments, the metallic foil is aluminum.

In certain embodiments, the microplate is labeled.

In certain embodiments, the one or more of said plurality of microwells contains a sufficient amount of said DNA end repair buffer to for perform said low volume DNA blunting and phosphorylation reactions.

In another aspect, the present disclosure provides a kit for use in low volume DNA blunting, phosphorylating, and adenylating reactions. The kit comprises: a first reagent plate of the present disclosure; a mixture of end repair enzymes comprising a DNA blunting enzyme at a concentration ranging from 0.2 U/μL to 1 U/μL and a DNA phosphorylating enzyme at a concentration ranging from 2 U/μL to 5 U/μL; and a second reagent plant of the present disclosure.

C. Reagent Plate for Use in Low Volume DNA Adaptor Ligation Reactions

In another aspect, the present disclosure provides a reagent plate for use in low volume DNA adaptor ligation reactions with adaptors. As used herein, this reagent plate is referred to as the “third reagent plate.” The reagent plate has at least one well containing a DNA ligation buffer for use in said low volume DNA adaptor ligation reactions with adaptors. In one embodiment, the DNA ligation buffer comprises a high concentration DNA ligation buffer mixture comprising: (i) Tris-HCl at a concentration ranging from 25 mM to 250 mM at a pH of 7.5 to 8.0; (ii) MgCl2 at a concentration ranging from 2.5 mM to 25 mM; (iii) DTT at a concentration ranging from 2.5 mM to 12.5 mM; (iv) ATP at a concentration ranging from 1.25 mM to 6.25 mM; and (v) PEG 6000 at a concentration ranging from 10 percent to 25 percent.

The high concentration DNA ligation buffer mixture, when provided at a volume ranging from 10 μL to 20 μL, is suitable for performing low volume adaptor ligation reactions of sample DNA fragments with a mixture of ligation enzymes at a concentration ranging from 80 c. U/μL to 200 c. U/μL, said mixture of ligation enzymes being provided at a volume ranging from 2.5 μL to 5 μL, and said adapters being provided at a volume ranging from 2.5 μL to 5 μL.

In certain embodiments, the high concentration DNA ligation buffer mixture comprises: (i) Tris-HCl at a concentration of about 50 mM at a pH of about 7.6; (ii) MgCl2 at a concentration of about 25 mM; (iii) DTT at a concentration of about 2.5 mM; (iv) ATP at a concentration of about 5 mM; and (v) PEG 6000 at a concentration of about 17.5 percent.

In certain embodiments, the high concentration DNA ligation buffer mixture, when provided at a volume of about 20 μL, is suitable for performing low volume DNA adaptor ligation reactions containing 20 μL of the sample DNA fragments, 5 μL of the mixture of ligation enzymes, and 5 μL of the adaptors, thereby resulting in a total volume of 50 μL during the performance of the low volume adaptor ligation reactions in the single container.

In certain embodiments, the 5 μL mixture of ligation enzymes is provided at a concentration of about 127 c. U/μL.

In certain embodiments, the ligation enzyme is selected from the group consisting of T4 DNA ligase, T3 DNA Ligase, and T7 DNA Ligase.

In certain embodiments, the reagent plate further comprises at least one component selected from the group consisting of Triton X-100, glycerol, NP 40, EDTA, Tween 20, and variants thereof.

In certain embodiments, the DNA ligation buffer is suitable for high throughput automated low volume DNA adaptor ligation reactions.

In certain embodiments, the DNA ligation buffer is suitable for low volume DNA ligation reactions that do not require a thermocycler.

In certain embodiments, the reagent plate is a microplate comprising a plurality of microwells, wherein each microwell corresponds to a separate, single container.

In certain embodiments, the microplate has a physical size that conforms to an industry standard.

In certain embodiments, the microplate includes any one of 96, 384, 1536, 3456 or 9600 microwells.

In certain embodiments, the microwells are arranged in a rectangular array.

In certain embodiments, the microplate is a standard 384-microwell microplate.

In certain embodiments, the microplate is made of any one of polystyrene and polypropylene.

In certain embodiments, the microplate is sealed.

In certain embodiments, the microplate is sealed with a metallic foil.

In certain embodiments, the metallic foil is piercable.

In certain embodiments, the metallic foil can be removed by peeling.

In certain embodiments, the metallic foil is aluminum.

In certain embodiments, the microplate is labeled.

In certain embodiments, the one or more of said plurality of microwells contains a sufficient amount of said DNA end repair buffer to for perform said low volume DNA blunting and phosphorylation reactions.

In another aspect, the present disclosure provides a kit for use in low volume DNA ligation reactions. The kit comprises a third reagent plate of the present disclosure and a mixture of ligation enzymes at a concentration ranging from 80 c. U/μL to 200 c. U/μL.

In another aspect, the present disclosure provides a kit for use in low volume DNA blunting, phosphorylating, adenylating, and ligation reactions. The kit comprises: a first reagent plate of the present disclosure; a mixture of end repair enzymes comprising a DNA blunting enzyme at a concentration ranging from 0.2 U/μL to 1 U/μL and a DNA phosphorylating enzyme at a concentration ranging from 2 U/μL to 10 U/μL; a second reagent plate of the present disclosure; a third reagent plate of the present disclosure; and a mixture of ligation enzymes.

### EXAMPLES

The present invention is described in further detail in the following examples which are not in any way intended to limit the scope of the invention as claimed. The attached Figures are meant to be considered as integral parts of the specification and description of the invention. All references cited are herein specifically incorporated by reference for all that is described therein. The following examples are offered to illustrate, but not to limit the claimed invention.

### Example 1

### Sequencing Library Preparation in Small Well Format

A process for preparing a library was designed that does not require a thermocycler and where the reaction volumes are low enough to be performed in a 384 well plate, therefore making it easy for high throughput automation.

One embodiment of this process is illustrated in FIG. 6 and is further described below.

End repair is performed by mixing the following: 20 uL of input DNA; 5 uL of custom 6× end repair buffer; and 5 uL of custom end repair enzyme mix. The 30 uL end repair reaction is incubated in room temperature for 30 minutes. The recipe for the custom 6× end repair buffer is shown in Table 1 and the recipe for the custom end repair enzyme mix is shown in Table 2, below:

TABLE 1
6X End Repair Buffer

| Component | 6X Condition |
|---|---|
| Water | |
| 1M Tris-HCl, pH 7.6 | 300 mM |
| 5M NaCl | 300 mM |
| 1M MgCl2 | 60 mM |
| 1M DTT | 30 mM |
| 100 mM ATP | 6 mM |
| 25 mM dNTP | 1.5 mM |

TABLE 2
End Repair Enzyme

| Component | 1X Condition |
|---|---|
| Enzyme Dilution | |
| Buffer | |
| T4 DNA Polymerase | 3 U |
| T4 PNK | 10 U |

A SPRIwork cleanup is performed to clean up the DNA. A 2:1 bead mix-to-sample ratio is used, which yields a 90 uL of total volume. After washing and drying the DNA bound beads, the beads are not eluted with elution buffer.

An A-tailing reaction is performed by adding 20 uL of dA-tailing mix directly to the DNA-bound beads. The 20 uL dA-tailing reaction is incubated in room temperature or 37 C for 30 minutes. The recipe for the dA-tailing mix is shown in Table 3, below:

TABLE 3
dA Tail Mix

| Component | 1X Condition |
|---|---|
| Water | |
| 1M Tris-HCl (pH 8.0) | 20 mM |
| 5M NaCl | 50 mM |
| 1M MgCl2 | 10 mM |
| 1M DTT | 1 mM |
| 100 mM dATP | 0.2 mM |
| Klenow Exo− (5U/uL) | 7.5 U |

Ligation is performed by mixing the following directly into the dA-tailing reaction: 20 uL of custom 2.5× ligation buffer; 5 uL of custom ligation enzyme mix; and 5 uL of adapters (concentration depends on input concentration). The 50 uL ligation reaction is incubated in room temperature for 15 minutes. The recipe for the custom 2.5× ligation buffer is shown in Table 4 and the recipe for the custom ligation enzyme mix is shown in Table 5, below:

TABLE 4
2.5X Ligation Buffer

| Component | 2.5X Condition |
|---|---|
| Water | |
| 1M Tris-HCl, pH 7.6 | 50 mM |
| 1M MgCl2 | 25 mM |
| 1M DTT | 2.5 mM |
| 100 mM ATP | 5 mM |
| 50% PEG 6000 | 17.5% |

TABLE 5
Ligation Enzyme

| Component | 1X Condition |
|---|---|
| Enzyme Dilution | |
| Buffer | |
| T4 DNA Ligase | 633 c. U |

A SPRIwork cleanup is performed to clean up the DNA. A 0.8:1 bead mix-to-sample ratio is used, which yields a 90 uL of total volume. After washing and drying the DNA bound beads, the beads are eluted in any desired volume.

Unless defined otherwise herein, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Marham, THE HARPER COLLINS DICTIONARY OF BIOLOGY, Harper Perennial, N.Y. (1991) provide one of skill with a general dictionary of many of the terms used in this invention. Although any methods and materials similar or equivalent to those described herein can be used in the practice or testing of the present invention, the preferred methods and materials are described. Practitioners are particularly directed to Sambrook et al., 1989, and Ausubel F M et al., 1993, for definitions and terms of the art. It is to be understood that this invention is not limited to the particular methodology, protocols, and reagents described, as these may vary.

Numeric ranges are inclusive of the numbers defining the range. The term about is used herein to mean plus or minus ten percent (10%) of a value. For example, “about 100” refers to any number between 90 and 110.

Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention, which can be had by reference to the specification as a whole. Accordingly, the terms defined immediately below are more fully defined by reference to the specification as a whole.

All patents and publications, including all sequences disclosed within such patents and publications, referred to herein are expressly incorporated by reference.

Other advantages which are obvious and which are inherent to the disclosure will be evident to one skilled in the art. It will be understood that certain features and sub-combinations are of utility and may be employed without reference to other features and sub-combinations. This is contemplated by and is within the scope of the claims. Since many possible embodiments may be made of the disclosure without departing from the scope thereof, it is to be understood that all matter herein set forth or shown in the accompanying drawings is to be interpreted as illustrative and not in a limiting sense.

1. A DNA end repair buffer for use in low volume DNA blunting and phosphorylating reactions, said buffer comprising:
a high concentration DNA end repair buffer mixture comprising: (i) deoxynucleoside triphosphates at a concentration ranging from 1 mM to 2.5 mM; (ii) Tris-HCl at a concentration ranging from 150 mM to 450 mM at a pH of 7.5 to 8.0; (iii) NaCl at a concentration ranging from 60 mM to 300 mM; (iv) MgCl₂ at a concentration ranging from 6 mM to 60 mM; (v) DTT at a concentration ranging from 6 mM to 30 mM; and (vi) ATP at a concentration ranging from 6 mM to 15 mM,
wherein said high concentration DNA end repair buffer mixture, when provided at a volume ranging from 2.5 μL to 5 μL, is suitable for performing low volume blunting and phosphorylating reactions of sample DNA fragments with a mixture of end repair enzymes in a single container, wherein said sample of DNA fragments is provided at a volume ranging from 10 μL to 20 μL, and
wherein said mixture of end repair enzymes comprises a DNA blunting enzyme at a concentration ranging from 0.2 U/μL, to 1.0 U/μL, and a DNA phosphorylating enzyme at a concentration ranging from 2.0 U/μL, to 5.0 U/μL said mixture of end repair enzymes being provided at a volume ranging from 2.5 μL to 5 μL.

2. The buffer according to claim 1, wherein said deoxynucleoside triphosphates comprise dATP, dCTP, dTTP, and dGTP, where the concentration of dATP ranges from 1 mM to 2.5 mM, the concentration of dCTP ranges from 1 mM to 2.5 mM, the concentration of dTTP ranges from 1 mM to 2.5 mM, and the concentration of dGTP ranges from 1 mM to 2.5 mM.

3. (canceled)

4. The buffer according to claim 1, wherein the high concentration DNA end repair buffer mixture comprises: (i) deoxynucleoside triphosphates at a concentration of about 1.5 mM; (ii) Tris-HCl at a concentration of about 300 mM at a pH of about 7.6; (iii) NaCl at a concentration of about 300 mM; (iv) MgCl₂ at a concentration of about 60 mM; (v) DTT at a concentration of about 30 mM; and (vi) ATP at a concentration of about 6 mM.

5. The buffer according to claim 1, wherein the high concentration DNA end repair buffer mixture, when provided at a volume of about 5 μL, is suitable for performing low volume blunting and phosphorylating reactions containing 20 μL of the sample DNA fragments and 5 μL of the mixture of end repair enzymes, thereby resulting in a total volume of 30 μL during the performance of the low volume blunting and phosphorylating reactions in the single container.

6. (canceled)

7. The buffer according to claim 1, wherein the DNA blunting enzyme is selected from the group consisting of T4 DNA polymerase, T7 DNA polymerase, and DNA Polymerase I, Large (Klenow) Fragment.

8. The buffer according to claim 1, wherein the DNA phosphorylating enzyme is selected from the group consisting of T4 polynucleotide kinase, and variants thereof.

9. The buffer according to claim 1 further comprising at least one component selected from the group consisting of Triton X-100, glycerol, NP 40, EDTA, Tween 20, and variants thereof.

10. The buffer according to claim 1, wherein said single container is a microwell of a microplate including any one of 96, 384, 1536, 3456 or 9600 microwells.

11. The buffer according to claim 1, wherein the DNA end repair buffer is suitable for high throughput automated low volume DNA blunting and phosphorylating reactions.

12. The buffer according to claim 1, wherein the DNA end repair buffer is suitable for low volume DNA blunting and phosphorylating reactions that do not require a thermocycler.

13. A kit for use in low volume DNA blunting and phosphorylating reactions, comprising:
the DNA end repair buffer according to claim 1; and
a mixture of end repair enzymes comprising a DNA blunting enzyme at a concentration ranging from 0.2 U/μL, to 1 U/μL, and a DNA phosphorylating enzyme at a concentration ranging from 2 U/μL, to 5 U/μL.

14. A DNA adenylating buffer for use in a low volume DNA adenylating reaction, said buffer comprising:
a high concentration DNA adenylating buffer mixture comprising: (i) Tris-HCl at a concentration ranging from 10 mM to 100 mM at a pH of 7.5 to 8.5; (ii) NaCl at a concentration ranging from 10 mM to 50 mM; (iii) MgCl₂ at a concentration ranging from 1 mM to 10 mM; (iv) DTT at a concentration ranging from 1 mM to 5 mM; (v) dATP at a concentration ranging from 0.1 mM to 0.5 mM; and (vi) Klenow fragment at a concentration ranging from 1 U/μL, to 10 U/μL,
wherein said high concentration DNA adenylating buffer mixture, when provided at a volume ranging from 5 μL to 20 μL, is suitable for performing low volume adenylating reactions of sample DNA fragments in a single container.

15. The buffer according to claim 14, wherein the high concentration DNA adenylating buffer mixture comprises: (i) Tris-HCl at a concentration of about 20 mM at a pH of about 8.0; (ii) NaCl at a concentration of about 50 mM; (iii) MgCl₂ at a concentration of about 10 mM; (iv) DTT at a concentration of about 1 mM; (v) dATP at a concentration of about 0.2 mM; and (vi) Klenow fragment at a concentration of about 0.375 U/μL.

16-20. (canceled)

21. A kit for use in low volume DNA blunting, phosphorylating, and adentylating reactions, comprising:
the DNA end repair buffer according to claim 1;
a mixture of end repair enzymes comprising a DNA blunting enzyme at a concentration ranging from 0.2 U/μL, to 1 U/μL, and a DNA phosphorylating enzyme at a concentration ranging from 2 U/μL, to 5 U/μL; and
a DNA adenylating buffer comprising a high concentration DNA adenylating buffer mixture comprising:
(i) Tris-HCl at a concentration ranging from 10 mM to 100 mM at a pH of 7.5 to 8.5;
(ii) NaCl at a concentration ranging from 10 mM to 50 mM;
(iii) MgCl₂ at a concentration ranging from 1 mM to 10 mM;
(iv) DTT at a concentration ranging from 1 mM to 5 mM;
(v) dATP at a concentration ranging from 0.1 mM to 0.5 mM; and
(vi) Klenow fragment at a concentration ranging from 1 U/μL, to 10 U/μL,
wherein said high concentration DNA adenylating buffer mixture, when provided at a volume ranging from 5 μL to 20 μL, is suitable for performing low volume adenylating reactions of sample DNA fragments in a single container.

22. A DNA ligation buffer for use in low volume DNA adaptor ligation reactions with adaptors, said buffer comprising:
a high concentration DNA ligation buffer mixture comprising: (i) Tris-HCl at a concentration ranging from 25 mM to 250 mM at a pH of 7.5 to 8.0; (ii) MgCl₂ at a concentration ranging from 2.5 mM to 25 mM; (iii) DTT at a concentration ranging from 2.5 mM to 12.5 mM; (iv) ATP at a concentration ranging from 1.25 mM to 6.25 mM; and (v) PEG 6000 at a concentration ranging from 10 percent to 25 percent,
wherein said high concentration DNA ligation buffer mixture, when provided at a volume ranging from 10 μL to 20 μL, is suitable for performing low volume adaptor ligation reactions of sample DNA fragments with a mixture of ligation enzymes at a concentration ranging from 80 c. U/μL, to 200 c. U/μL said mixture of ligation enzymes being provided at a volume ranging from 2.5 μL to 5 μL, and said adapters being provided at a volume ranging from 2.5 μL, to 5 μL.

23. The buffer according to claim 22, wherein the high concentration DNA ligation buffer mixture comprises: (i) Tris-HCl at a concentration of about 50 mM at a pH of about 7.6; (ii) MgCl₂ at a concentration of about 25 mM; (iii) DTT at a concentration of about 2.5 mM; (iv) ATP at a concentration of about 5 mM; and (v) PEG 6000 at a concentration of about 17.5 percent.

24. The buffer according to claim 22, wherein the high concentration DNA ligation buffer mixture, when provided at a volume of about 20 μL, is suitable for performing low volume DNA adaptor ligation reactions containing 20 μL of the sample DNA fragments, 5 μL of the mixture of ligation enzymes, and 5 μL of the adaptors, thereby resulting in a total volume of 50 μL during the performance of the low volume adaptor ligation reactions in the single container.

25. (canceled)

26. The buffer according to claim 22, wherein the ligation enzyme is selected from the group consisting of T4 DNA ligase, T3 DNA Ligase, and T7 DNA Ligase.

27-30. (canceled)

31. A kit for use in low volume DNA ligation reactions, comprising:
the DNA ligation buffer according to claim 22; and
a mixture of ligation enzymes at a concentration ranging from 80 c. U/μL to 200 c. U/μL.

32. A kit for use in low volume DNA blunting, phosphorylating, adenylating, and ligation reactions, comprising:
(a) the DNA end repair buffer according to claim 1;
(b) a mixture of end repair enzymes comprising a DNA blunting enzyme at a concentration ranging from 0.2 U/μL to 1 U/μL and a DNA phosphorylating enzyme at a concentration ranging from 2 U/μL to 10 U/μL;
(a) a DNA adenylating buffer comprising a high concentration DNA adenylating buffer mixture comprising:
(i) Tris-HCl at a concentration ranging from 10 mM to 100 mM at a pH of 7.5 to 8.5;
(ii) NaCl at a concentration ranging from 10 mM to 50 mM;
(iii) MgCl₂ at a concentration ranging from 1 mM to 10 mM;
(iv) DTT at a concentration ranging from 1 mM to 5 mM;
(v) dATP at a concentration ranging from 0.1 mM to 0.5 mM; and
(vi) Klenow fragment at a concentration ranging from 1 U/μL to 10 U/μL,
wherein said high concentration DNA adenylating buffer mixture, when provided at a volume ranging from 5 μL to 20 μL, is suitable for performing low volume adenylating reactions of sample DNA fragments in a single container;
(d) a DNA ligation buffer comprising a high concentration DNA ligation buffer mixture comprising:
(i) Tris-HCl at a concentration ranging from 25 mM to 250 mM at a pH of 7.5 to 8.0;
(ii) MgCl₂ at a concentration ranging from 2.5 mM to 25 mM;
(iii) DTT at a concentration ranging from 2.5 mM to 12.5 mM;
(iv) ATP at a concentration ranging from 1.25 mM to 6.25 mM; and
(v) PEG 6000 at a concentration ranging from 10 percent to 25 percent,
wherein said high concentration DNA ligation buffer mixture, when provided at a volume ranging from 10 μL to 20 μL, is suitable for performing low volume adaptor ligation reactions of sample DNA fragments with a mixture of ligation enzymes at a concentration ranging from 80 c. U/μL, to 200 c. U/μL, said mixture of ligation enzymes being provided at a volume ranging from 2.5 μL to 5 μL, and said adapters being provided at a volume ranging from 2.5 μL, to 5 μL; and
(e) a mixture of ligation enzymes.
